(19) United States
(12) Patent Application Publication
(10) Pub. No.: US 2026/0315283 A1
(43) Pub. Date: Oct. 15, 2026

(54) EUSTACHIAN TUBE THERAPEUTIC SUBSTANCE DELIVERY DEVICE

(71) Applicant: AbsorbENT, LLC, Houston, TX (US)

(72) Inventors: Joseph EDMONDS, Houston, TX (US); Abel SMERICA, Montgomery, TX (US)

(21) Appl. No.: 19/174,763

(22) Filed: Apr. 9, 2025

(51) Int. Cl. A61F 11/20

(52) U.S. Cl. CPC A61F 11/202; A61F 2210/0004; A61F 2220/0016; A61F 2250/0067

(57) ABSTRACT

A eustachian tube therapeutic substance delivery device is described. The device includes a body suitable for implantation into a patient's eustachian tube. The body is formed of a resorbable material. The body carries the therapeutic substance so that the therapeutic substance is discharged as the body is resorbed.

### BACKGROUND

The eustachian tube is a narrow canal that connects the middle ear to the nasopharynx, the upper part of the throat behind the nose. This structure plays a crucial role in maintaining the proper function of the ear by equalizing pressure between the middle ear and the external environment, ensuring that sound is transmitted effectively. Additionally, the eustachian tube serves as a drainage pathway for mucus and other secretions from the middle ear, preventing the buildup of fluid that could lead to infections and hearing issues. Structurally, it consists of a bony portion near the middle ear and a fibrocartilaginous portion closer to the throat. The latter is flexible, allowing the tube to open and close to regulate air pressure and fluid drainage.

When the eustachian tube fails to function properly, it leads to a condition known as eustachian tube dysfunction, which impairs the ear's ability to regulate pressure and clear accumulated fluids. As a result, individuals with this dysfunction frequently experience middle ear infections, known as otitis media, which can cause pain, temporary hearing loss, and discomfort. The accumulation of fluid in the middle ear further exacerbates these issues, sometimes leading to persistent hearing impairment. Pressure imbalances within the ear, particularly during changes in altitude such as flying or diving, can also cause significant pain and damage, a condition referred to as barotrauma. Chronic dysfunction may contribute to long-term complications, including permanent hearing loss if the condition is left untreated.

For many years, the standard approach to managing middle ear infections and eustachian tube dysfunction has involved the insertion of ventilation tubes, commonly known as ear tubes, through a procedure called myringotomy. This involves making a small incision in the eardrum to drain fluid, followed by the placement of a tiny tube that allows continuous ventilation and pressure regulation. Myringotomy with tube insertion has been particularly common in children, who are more prone to ear infections due to the immature development of their eustachian tubes. In addition to surgical intervention, drug therapies such as antibiotics for bacterial infections, decongestants to reduce swelling in the nasal passages, and antihistamines in cases of allergy-related inflammation have also been widely used to manage symptoms and prevent recurring infections.

Despite the effectiveness of these treatments in managing symptoms, they do not address the underlying dysfunction of the eustachian tube itself. In recent years, an alternative treatment known as balloon dilation of the eustachian tube has gained attention as a potential method for restoring its natural function. This procedure involves the insertion of a small catheter with a balloon at its tip through the nasal passage and into the eustachian tube. Once properly positioned, the balloon is inflated to gently stretch the fibrocartilaginous portion of the tube, widening its diameter and potentially improving its ability to open and close naturally. After a short period, the balloon is deflated and removed, with the goal of enhancing the tube's function and allowing for better drainage and pressure regulation.

Although balloon dilation presents a promising, minimally invasive option for individuals suffering from chronic eustachian tube dysfunction, its long-term efficacy remains an area of ongoing study. The structure of the eustachian tube, particularly its flexible fibrocartilaginous portion, raises questions about whether dilation provides a lasting solution or merely temporary relief. While some studies suggest that the procedure can reduce the risk of repeated ear tube placement or medication, it is not yet clear whether the benefits persist over time.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIGS. 1A-1C depict a straight cylinder stent eustachian tube therapeutic substance delivery device, in accordance with some embodiments.

FIGS. 2A-2C depict a tapered conical stent eustachian tube therapeutic substance delivery device, in accordance with some embodiments.

FIGS. 3A-3G depict an hourglass stent eustachian tube therapeutic substance delivery device, in accordance with some embodiments.

FIGS. 4A-4C depict a helical stent eustachian tube therapeutic substance delivery device, in accordance with some embodiments.

FIGS. 5A-5C depict a mesh stent eustachian tube therapeutic substance delivery device, in accordance with some embodiments.

FIG. 6A-6D depict a segmented rings stent eustachian tube therapeutic substance delivery device, in accordance with some embodiments.

FIG. 7A-7C depict filament stent eustachian tube therapeutic substance delivery devices, in accordance with some embodiments.

FIGS. 8A-8C depict a ribbed eustachian tube therapeutic substance delivery device, in accordance with some embodiments.

FIGS. 9A-9C depict a granules eustachian tube therapeutic substance delivery device, in accordance with some embodiments.

FIGS. 10A-10C depict a ventilation tube eustachian tube therapeutic substance delivery device, in accordance with some embodiments.

FIG. 11 depicts a eustachian tube therapeutic substance delivery insertion tool, in accordance with some embodiments.

FIG. 12 depicts a eustachian tube therapeutic substance delivery implantation, in accordance with some embodiments.

FIGS. 13A-13C depict implanted eustachian tube therapeutic substance delivery devices, in accordance with some embodiments.

### DETAILED DESCRIPTION

The following disclosure provides many different embodiments, or examples, for implementing different features of the provided subject matter. Specific examples of components, values, operations, materials, arrangements, or the like, are described below to simplify the present disclosure. These are, of course, merely examples and are not intended to be limiting. Other components, values, operations, materials, arrangements, or the like, are contemplated. For example, the formation of a first feature over or on a second feature in the description that follows may include embodiments in which the first and second features are formed in direct contact and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Further, spatially relative terms, such as “beneath,” “below,” “lower,” “above,” “upper” and the like, may be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus may be otherwise oriented (rotated 90 degrees or at other orientations) and the spatially relative descriptors used herein may likewise be interpreted accordingly.

The eustachian tube therapeutic substance delivery device increases the efficacy of a eustachian tube dilation procedure, potentially reducing complications or the potential for additional surgery.

The eustachian tube therapeutic substance delivery device, in accordance with an embodiment, is a physical body made from a biocompatible resorbable material and carries a therapeutic substance so that the substance is discharged as the stent is resorbed, to be implanted in the eustachian tube of a patient.

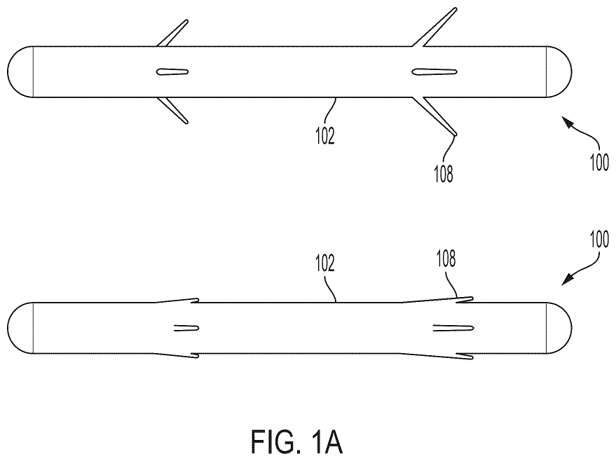

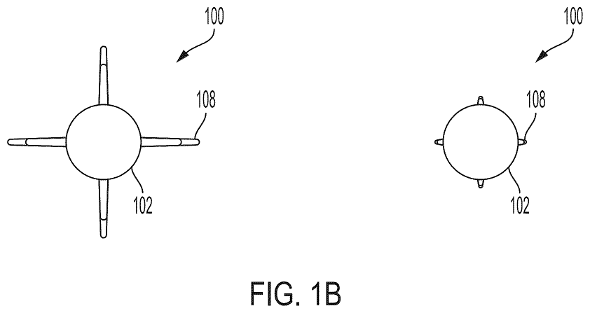

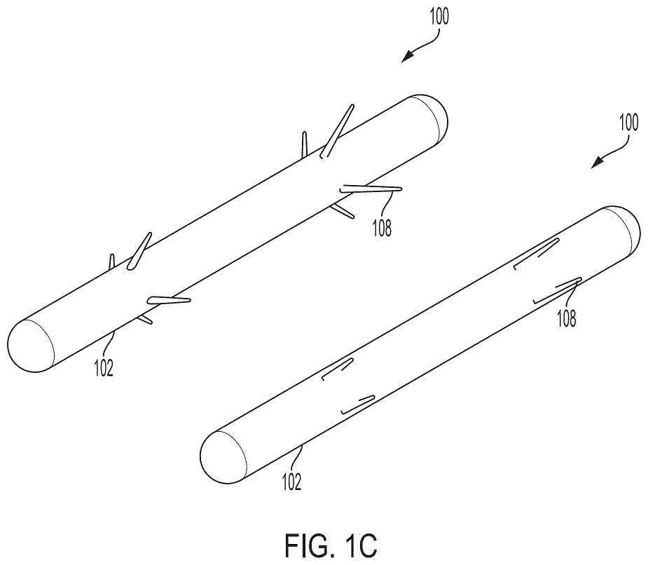

FIGS. 1A-1C depict, in accordance with an embodiment, a eustachian tube therapeutic substance delivery device 100. The eustachian tube therapeutic substance delivery device 100 has a body 102. In accordance with various embodiments, the body 102 is formed in a variety of shapes, sizes and configurations and is shown here as a straight cylinder. The body 102 may include one or more retention features 108. The body 102 is formed of a biocompatible resorbable material. The body 102 carries a therapeutic substance. The body 102 may be impregnated with the therapeutic substance. The body 102 may be coated with the therapeutic substance. The body 102 is sized appropriately for implantation into a patient's eustachian tube, typically about 2 millimeters in diameter and 20 millimeters in length. The body 102 is formed from a rigid, semi-rigid, or flexible resorbable material

### Configurations

In accordance with an embodiment, the body 102 of the straight cylinder stent eustachian tube therapeutic substance delivery device 100 has the shape of a straight cylinder. The straight cylinder stent body 102 is shaped similarly to traditional vascular or airway stents but sized appropriately to implantation in a patient's eustachian tube. The straight cylinder stent body 102 may insert easily into the eustachian tube and made to expand in diameter to engage the surface of the eustachian tube.

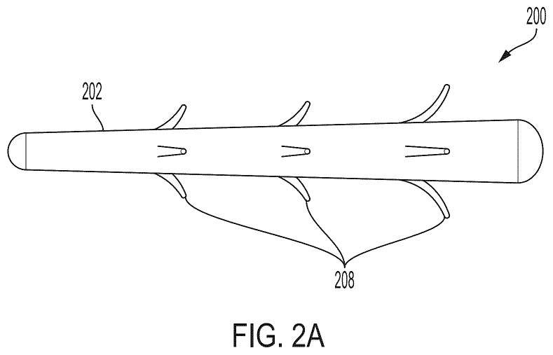

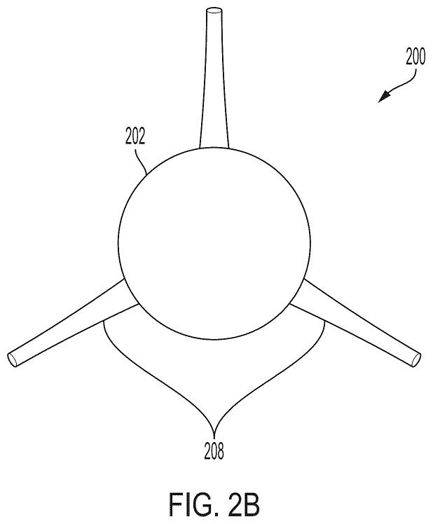

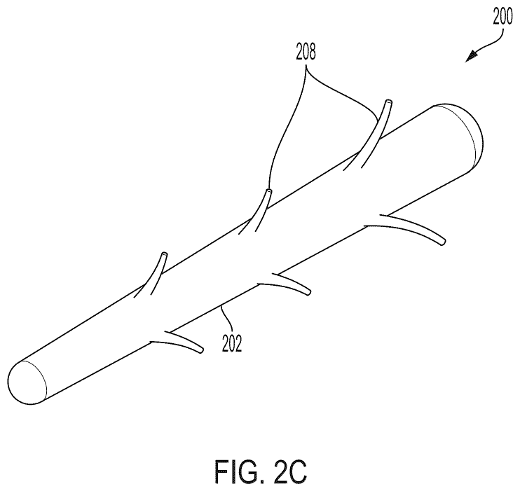

FIGS. 2A-2C depict, in accordance with an embodiment, a tapered conical stent eustachian tube therapeutic substance delivery device 200. The stent body 202 has the shape of a tapered cone so that the stent is generally conical. The tapered conical stent body 202 may be designed with a wider end to be positioned when implanted toward the nasopharynx and a narrower end positioned toward the middle ear. The tapered conical stent body 202 mimics the natural anatomy of the eustachian tube, reducing migration and improving fit. The body 202 may include one or more retention features 208.

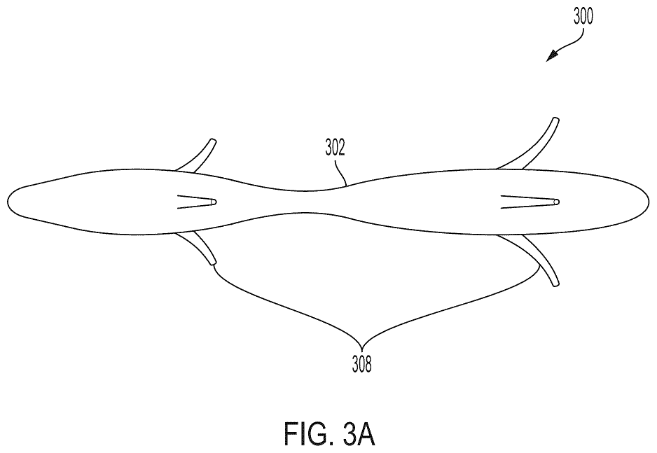

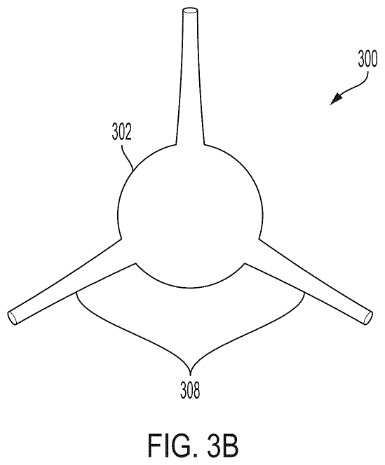

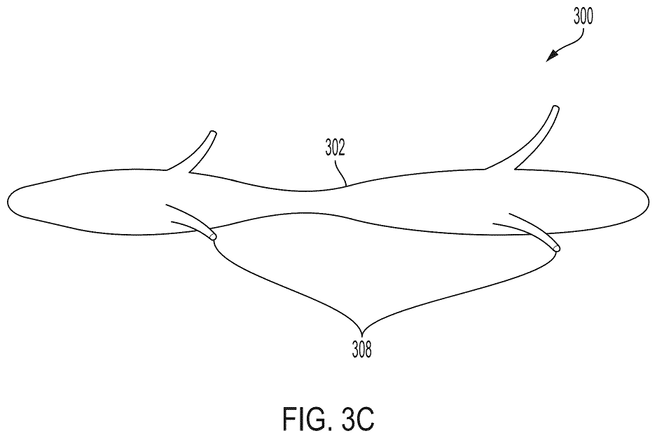

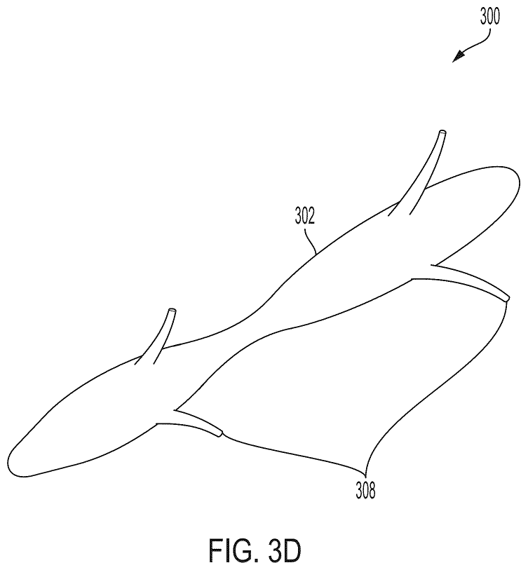

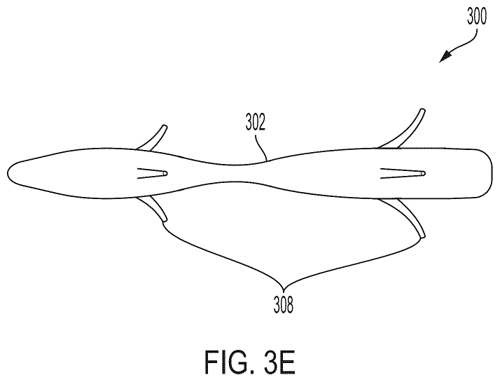

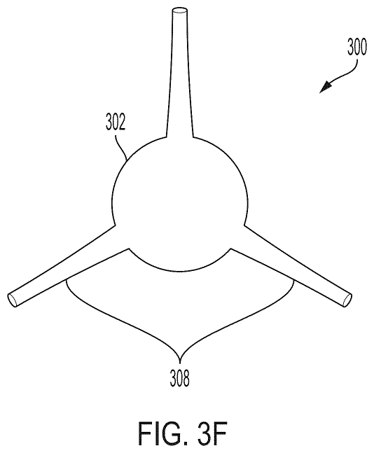

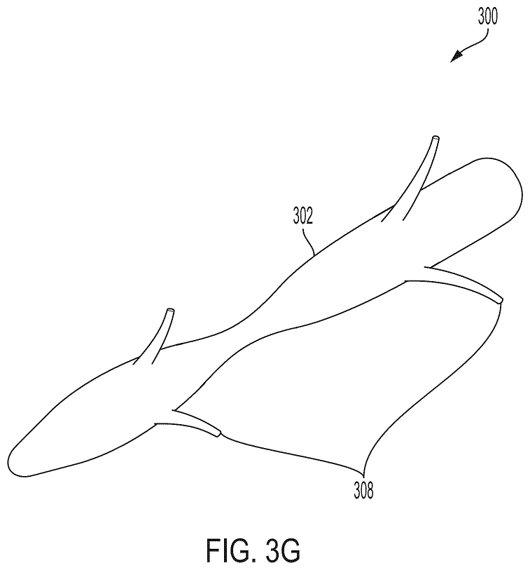

FIGS. 3A-3G depict, in accordance with an embodiment, an hourglass stent eustachian tube therapeutic substance delivery device 300. The stent body 302 has an hourglass shape, slightly narrower in the middle with flared ends. The hourglass shape prevents migration while maintaining an open lumen for drainage and ventilation. The eustachian tube is thereby able to open and close naturally. An hourglass stent body made using a mesh allows the delivery device 300 to flex in response to variations in the opening. The body 302 may include one or more retention features 308.

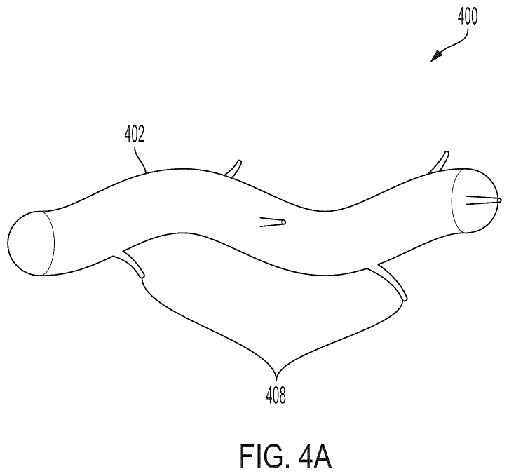

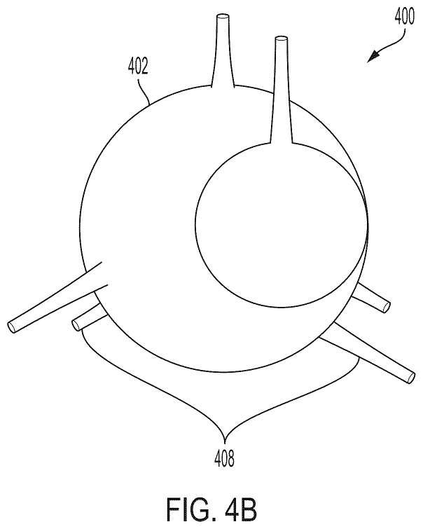

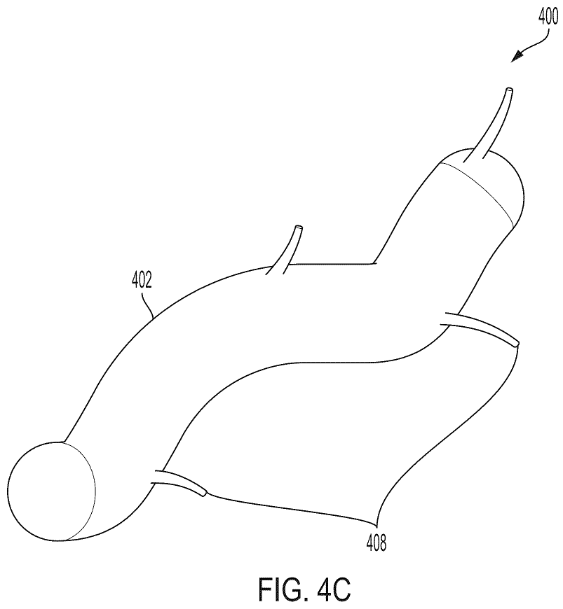

FIGS. 4A-4C depict, in accordance with an embodiment, a helical stent eustachian tube therapeutic substance delivery device 400. The stent body 402 has a helical or spiral shape. The helical shaped stent body 402 is a flexible, coiled structure that can expand and contract with tube movement. The helical shaped stent body 402 reduces the risk of complete blockage of the tube and allows for better mucociliary clearance and reduced irritation. The body 402 may include one or more retention features 408.

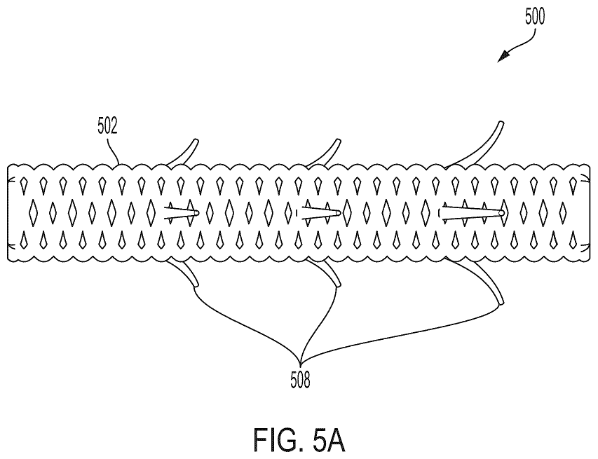

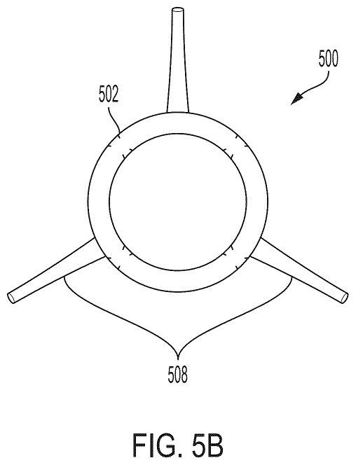

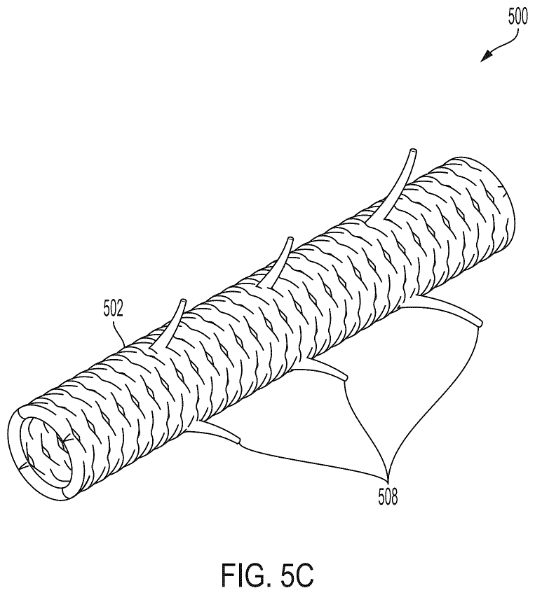

FIGS. 5A-5C depict, in accordance with an embodiment, a mesh stent eustachian tube therapeutic substance delivery device 500. The stent body 502 has a mesh or scaffold-based shape. The mesh stent body 502 has an open framework, similar to coronary stents, allowing mucus to pass through. The mesh stent body 502 provides mechanical support without completely obstructing normal function. The density of the mesh is completely variable. The mesh, in accordance with various embodiments, ranges from a very loose and thin mesh to allow movement, to a denser, thicker mesh for strength. The body 502 may include one or more retention features 508.

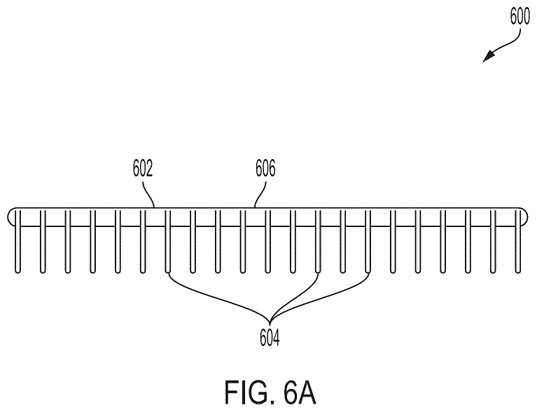

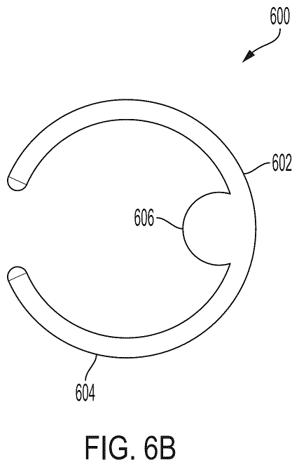

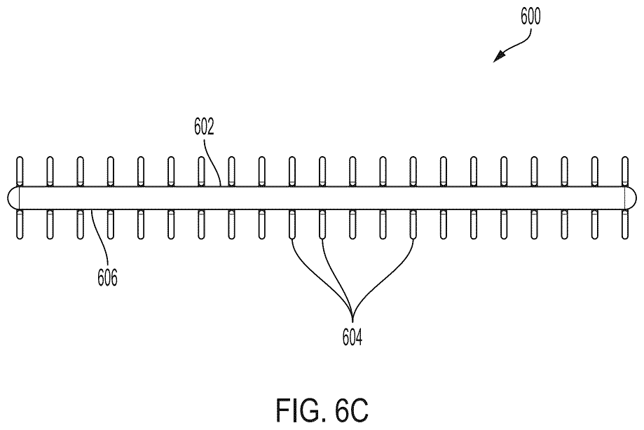

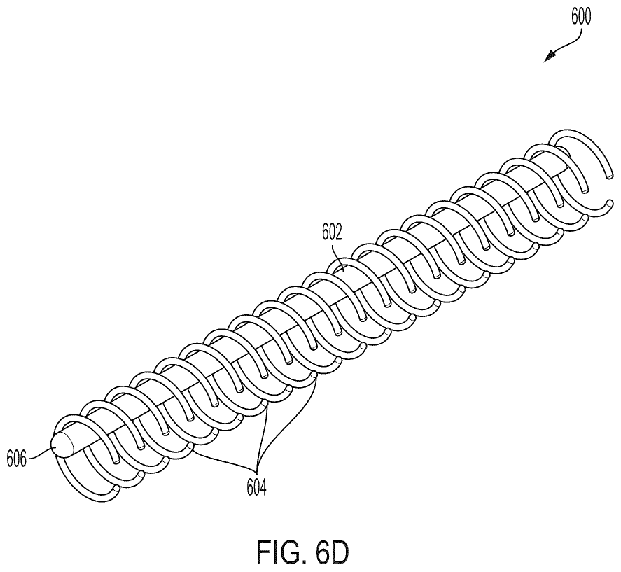

FIGS. 6A-6D depict, in accordance with an embodiment, a segmented ring stent eustachian tube therapeutic substance delivery device 600. The stent body 602 is segmented, formed with expandable rings 604: The segmented stent body 602 is composed of multiple linked segments 604 connected to a central spine 606. The segmented stent body 602 is flexible and prevents blockage while adapting to movements of the surrounding structures.

In accordance with several embodiments, the stent body 102 has a hybrid design. A hybrid stent body 102 may include a combination of shapes, such as a helical-tapered or mesh-hourglass, optimizing stability, airflow, and mucus clearance.

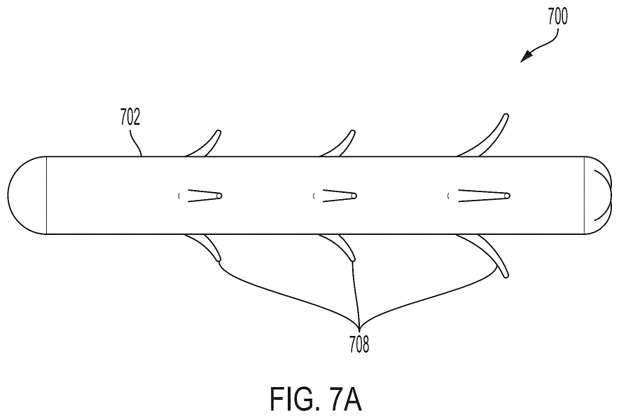

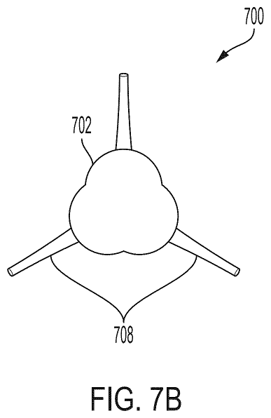

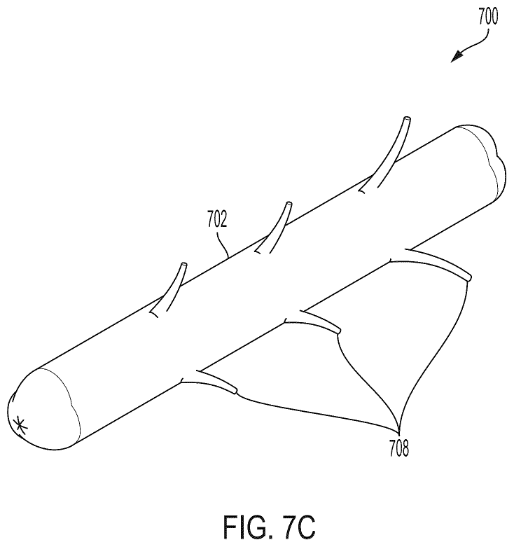

FIGS. 7A-7C depict, in accordance with an embodiment, a filament stent eustachian tube therapeutic substance delivery device 700. The stent body 702 is formed by fusing a plurality of filaments or rods, which may be fused at one location or along the entirety of the stent. The body 702 may include one or more retention features 708.

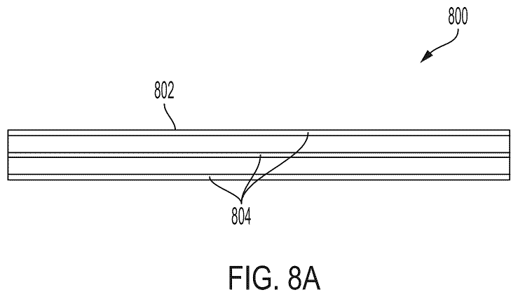

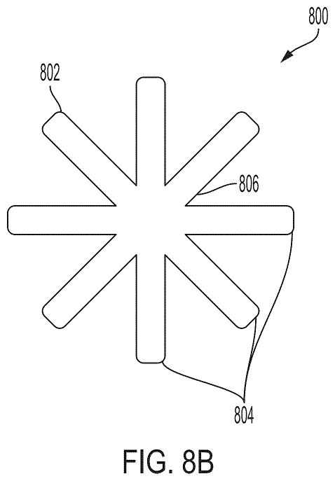

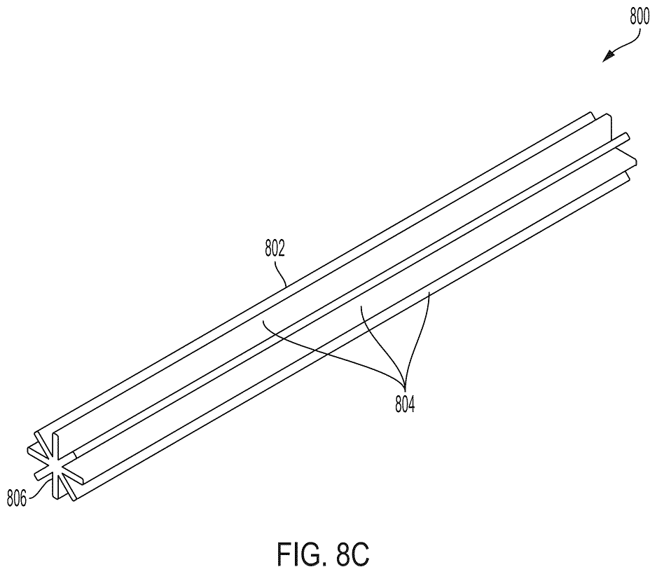

FIGS. 8A-8C depict, in accordance with an embodiment, a ribbed stent eustachian tube therapeutic substance delivery device 800. The stent body 802 is formed with an asterisk profile, with ribs 804 extending from a central core 806. The ribbed stent body 802 is manufactured using extrusion.

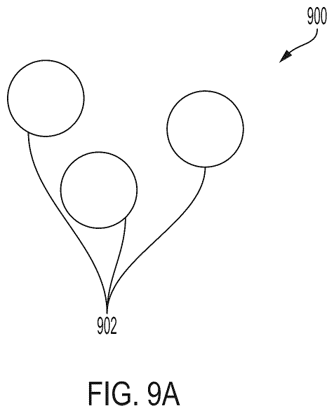

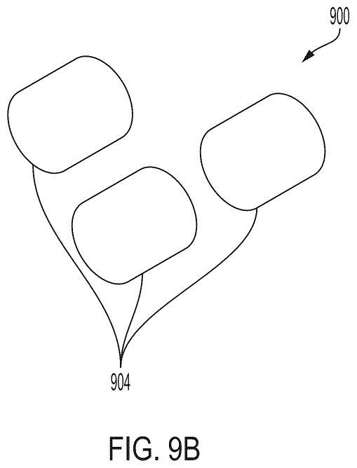

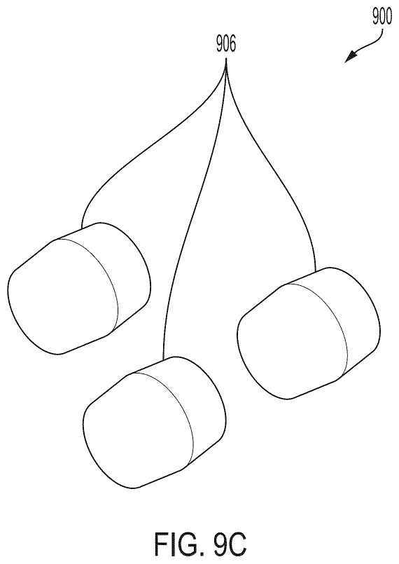

FIG. 9A-9C depict, in accordance with various embodiments, eustachian tube therapeutic substance delivery device granules 900. In accordance with an embodiment, the granules bodies 902 are spheres. In accordance with an embodiment, the granules bodies 904 are rod shaped. In accordance with an embodiment, the granules bodies 906 are barrel shaped. In accordance with an embodiment, other suitable shapes are used, as appropriate. In cases where a myringotomy has been performed, granules bodies 902, 904 or 906 are formed from a PLA/PLGA blend material with drug eluting properties. These granules are sized to allow placement in the middle ear via the perforated eardrum, typically about 1-2 millimeters in diameter.

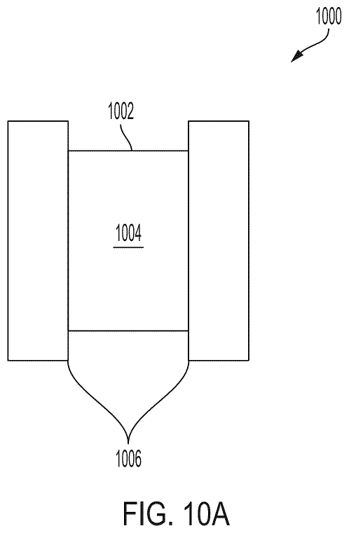

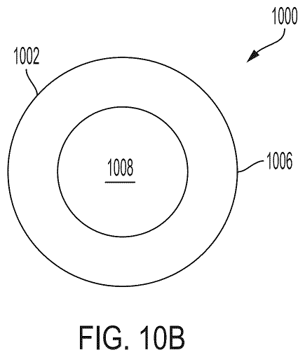

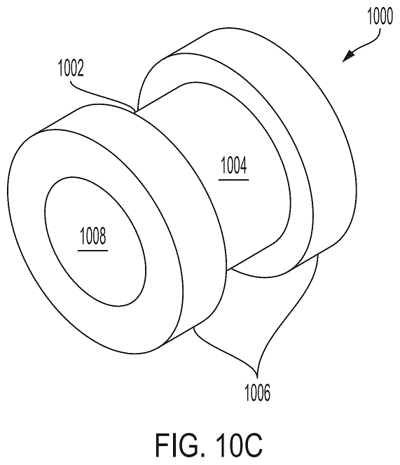

FIGS. 10A-10C depict, in accordance with an embodiment, a ventilation tube eustachian tube therapeutic substance delivery device 1000. The body 1002 forms a pressure equalizing ventilation tube. The resorbable pressure equalizing ventilation tube body 1002 is installed across the eardrum. In accordance with an embodiment, the body 1002 is formed of a PLA/PLGA blend material with drug eluting properties. The body 1002 is about 3-4 millimeters in diameter in the center section 1004 where it has contact with the eardrum, and about 3-5 millimeters at the outer edges 1006 to prevent the tube from falling from the eardrum. A hole 1008 of about 1-2 millimeters in the middle of the body 1002 provides ventilation to the middle ear.

### Retention Features

In accordance with various embodiments, the eustachian tube therapeutic substance delivery devices include retention features of various size, shape, and count. The retention features 108, 208, 308, 408, 508 and 708 extend from the bodies of the eustachian tube therapeutic substance delivery devices to maintain the device position frictionally within the eustachian tube and to prevent aspiration of the device. The retention features 108, 208, 308, 408, 508 and 708 may be bulges, protrusions, enlarged portions, cylindrical shapes or any other suitable addition or deformation to improve the stability of the stent within the patient's eustachian tube.

### Resorbable Material

The eustachian tube therapeutic substance delivery devices may be formed from a variety of resorbable materials or a combination of materials and resorbable material.

In accordance with an embodiment, the eustachian tube therapeutic substance delivery devices may be formed of polymers. The polymers may be selected from synthetic polymers and natural polymers or a combination of polymers.

In accordance with an embodiment, the eustachian tube therapeutic substance delivery device is formed of polylactic acid (PLA): PLA is widely used in biodegradable medical implants. PLA provides rigid support initially and degrades slowly, typically months to years. The PLA breakdown products are primarily lactic acid, which are metabolized naturally.

In accordance with an embodiment, the eustachian tube therapeutic substance delivery device is formed of polyglycolic acid (PGA) PGA degrades faster than PLA, typically weeks to months. PGA is used in absorbable sutures. PGA is hydrophilic, making it suitable for moist environments like the eustachian tube.

In accordance with an embodiment, the eustachian tube therapeutic substance delivery device is formed of polylactic-co-glycolic acid (PLGA). PLGA is a blend of PLA and PGA with tunable degradation rates and tunable flexibility. PLGA is common in drug delivery systems and biodegradable implants. PLGA has better mechanical properties and degradation control than PLA or PGA alone.

In accordance with an embodiment, the eustachian tube therapeutic substance delivery device is formed of polycaprolactone (PCL). PCL has an extremely slow degradation rate, typically years, but blending with PGA/PLA can speed up resorption. PCL is highly flexible and soft, which reduces irritation.

In accordance with an embodiment, the eustachian tube therapeutic substance delivery device is formed of polyhydroxyalkanoates (PHA). PHA are naturally occurring biopolymers. PHA has good biocompatibility with intermediate degradation time.

In accordance with an embodiment, the eustachian tube therapeutic substance delivery device is formed of chitosan, derived from chitin, a material found in shellfish. Chitosan has antimicrobial properties and promotes wound healing. Chitosan may be modified for controlled degradation.

In accordance with an embodiment, the eustachian tube therapeutic substance delivery device is formed of hyaluronic acid-based hydrogels. Hyaluronic acid-based hydrogels are naturally present in human tissue and provide temporary mechanical support while enhancing mucosal healing.

In accordance with an embodiment, the eustachian tube therapeutic substance delivery device is formed with magnesium-based bioabsorbable metals.

In accordance with an embodiment, the eustachian tube therapeutic substance delivery device is formed of magnesium (Mg) alloys. Mg alloys slowly dissolve in bodily fluids, leaving no permanent foreign material. Mg alloys are stronger than most polymers but still flexible.

In accordance with an embodiment, the eustachian tube therapeutic substance delivery device is formed of zinc (Zn) alloys. Zn alloys are degradable metals with better stability than pure magnesium. Zn alloys have a slower degradation rate than Mg, potentially making it more predictable.

In accordance with an embodiment, the eustachian tube therapeutic substance delivery device is formed of iron (Fe)-based bioabsorbable materials. Fe-based bioabsorbable materials have a much slower degradation than Mg or Zn.

In accordance with an embodiment, the eustachian tube therapeutic substance delivery device is formed of composite materials.

In accordance with an embodiment, the eustachian tube therapeutic substance delivery device is formed of PLGA+magnesium particles. This composite combines the flexibility of PLGA with the strength of magnesium. The degradation rate can be fine-tuned for specific applications.

In accordance with an embodiment, the eustachian tube therapeutic substance delivery device is formed of PGA/PLA blends with chitosan or hyaluronic acid. This composite enhances biocompatibility while maintaining mechanical integrity, reduces inflammation and promotes healing.

In accordance with an embodiment, the eustachian tube therapeutic substance delivery device is formed of silk fibroin-based bio-scaffolds. Derived from silk proteins, the silk fibroin-based bio-scaffolds provide structural support and resorbs naturally.

In accordance with an embodiment, the eustachian tube therapeutic substance delivery device has self-expanding memory. The eustachian tube therapeutic substance delivery device is made from a shape-memory alloy that expands when placed in the body. The nitinol-based stent body conforms to the tube's anatomy and self-adjusts to pressure changes.

### Therapeutic Substances

In accordance with several embodiments, the eustachian tube therapeutic substance delivery device carries a therapeutic substance. In accordance with various embodiments, the eustachian tube therapeutic substance delivery device is impregnated with one or more therapeutic substances. In accordance with various embodiments, the eustachian tube therapeutic substance delivery device is coated with one or more therapeutic substances.

In accordance with an embodiment, the eustachian tube therapeutic substance delivery device carries anti-inflammatory drugs. Since eustachian tube dysfunction often involves chronic inflammation, reducing swelling and improving tube function is a major goal. The impregnated anti-inflammatory drugs may include: corticosteroids, dexamethasone, mometasone, fluticasone, budesonide or any other suitable anti-inflammatory drug.

In accordance with an embodiment, the eustachian tube therapeutic substance delivery device carries non-steroidal anti-inflammatory drugs (NSAIDs). The impregnated NSAIDs may include: Ketorolac, Celecoxib, or any other suitable NSAIDs.

In accordance with an embodiment, the eustachian tube therapeutic substance delivery device carries mucolytics and/or surfactants: These drugs help thin mucus and improve drainage, which is essential for clearing the eustachian tube. The impregnated mucolytics and surfactants may include: N-Acetylcysteine (NAC), Erdosteine, Surfactant-based formulations, or any other suitable mucoytics or surfactants.

In accordance with an embodiment, the eustachian tube therapeutic substance delivery device carries antibiotics and/or antimicrobials. Since chronic or recurrent middle ear infections (otitis media) are common with ETD, an antimicrobial drug-eluting stent could help prevent bacterial colonization. The impregnated antibiotics and antimicrobials may include: broad-spectrum antibiotics, azithromycin, levofloxacin, clarithromycin, anti-biofilm agents, xylitol, gallium nitrate, antifungal agents, clotrimazole, miconazole, or any other suitable antibiotics or antimicrobials.

In accordance with an embodiment, the eustachian tube therapeutic substance delivery device carries vasodilators and/or anti-fibrotic agents. If chronic inflammation leads to fibrosis and scarring, these drugs could help maintain eustachian tube flexibility. The vasodilators and/or anti-fibrotic agents may include: bosentan, losartan, hyaluronic acid or any other suitable vasodilators and/or anti-fibrotic agents.

In accordance with at least one embodiment, eustachian tube therapeutic substance delivery device carries growth factors and tissue repair agents. To enhance mucosal healing and regeneration, controlled drug release of tissue-repairing factors could be useful. The growth factors and tissue repair agents may include: epidermal growth factor (EGF), fibroblast growth factor (FGF-2), vitamin A (Retinoic Acid Derivatives) or any other growth factors and tissue repair agents.

In accordance with an embodiment, the eustachian tube therapeutic substance delivery device carries local anesthetics and/or pain modulators. To reduce post-procedure discomfort, the stent could incorporate local anesthetics for a short-term effect. The local anesthetics and/or pain modulators may include: lidocaine, bupivacaine, capsaicin (low dose), or any other suitable local anesthetics and/or pain modulators.

Steroid therapy is chosen because of its ability to reduce inflammation and prevent scar formation in the dilated tube's epithelium. The release of the drug depends on the duration of the eustachian tube therapeutic substance delivery device's resorption, which would be designed to last from a few weeks to months, depending on the therapeutic effect desired.

The eustachian tube therapeutic substance delivery device carries steroid or other drugs including monoclonal antibody therapy, chemotherapy or antivirals for HPV infections, in a timed release to assist in the therapeutic effect. Being fully impregnated with a drug controls the release over a longer period of time vs. being coated in a drug, which would initially release quickly over a few days and then the drug therapy would end.

### Manufacture

The materials used to form the eustachian tube therapeutic substance delivery devices, for example PLA and PGA (or PLGA mix, etc.) in crystal or granular form are then melted, typically at a temperature of around 50-80 deg C. The molten material is mixed with therapeutic substances, such as medications, which are typically in the form of a powder, for example mometasone furoate powder, as a homogenous suspension while in the melted state. Then the mixture is extruded or mold injected into the desired shape.

The eustachian tube therapeutic substance delivery devices, in accordance with an embodiment, are coated in a therapeutic substance to provide a dose of a therapeutic substance for a relatively short duration.

### Methods of Use

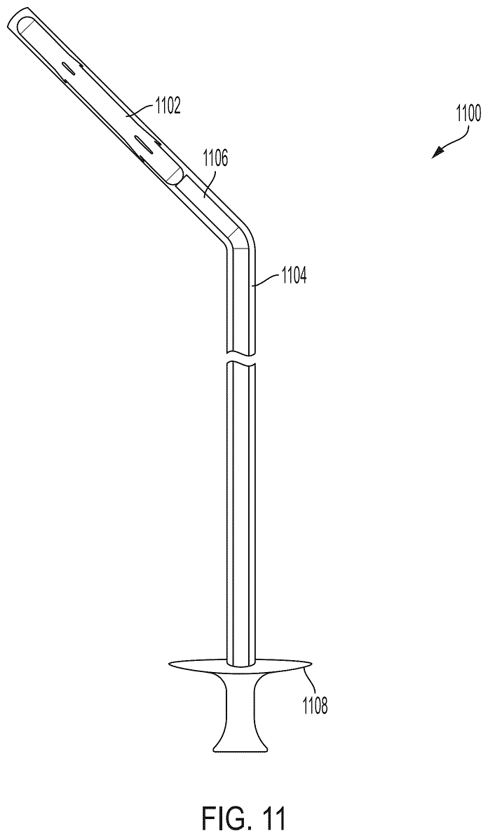

FIG. 11 depicts, in accordance with an embodiment, a eustachian tube therapeutic substance delivery device implantation tool 1100 is shown. The implantation tool 1100 includes an outer sheath 1104 and an inner member 1106 that moves within the outer sheath 1104 to apply pressure from a handle 1108 to a eustachian tube therapeutic substance delivery device 1002 for insertion into a patient's eustachian tube.

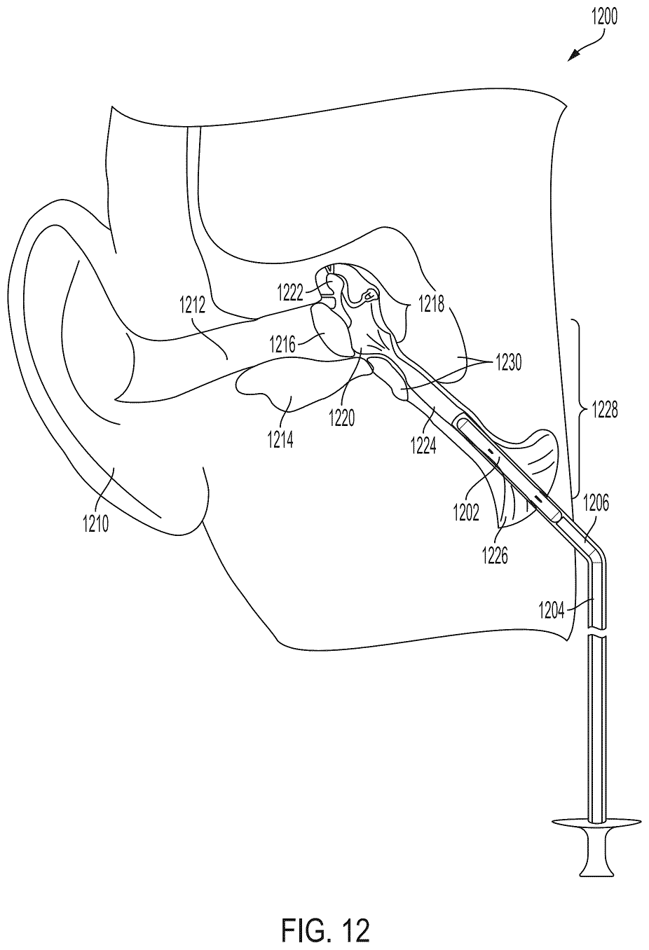

FIG. 12 depicts, in accordance with an embodiment, an implantation procedure 1200. The figure depicts the anatomy of a patient's eustachian tube pathway including the external ear 1210, the external auditory meatus 1212, the bony part of external auditory meatus 1214, the tympanic membrane 1216, the middle ear 1218, the tympanic cavity 1220, the auditory ossicles 1222, the eustachian tube 1224, the pharyngeal opening of the Eustachian tube surrounded by the torus tubarius 1226 and the nasopharynx 1228. The eustachian tube 1224 is within the bony part of the eustachian tube 1230.

The insertion tool 1204 includes an inner member 1206 to apply pressure to the eustachian tube drug eluting device 1202 placed within the insertion tool 1204. With direct visualization of the torus tubarius 1226 and the opening of the eustachian tube 1224 via nasal endoscopy, the eustachian tube drug eluting device insertion tool 1204 is positioned into the orifice. Using the insertion tool 1204, the drug eluting device 1202 is slowly pushed into the eustachian tube 1224 from the insertion tool 1204 using the inner member 1206 and the insertion tool 1204 is then removed leaving the drug eluting device 1202 in place within the eustachian tube 1224.

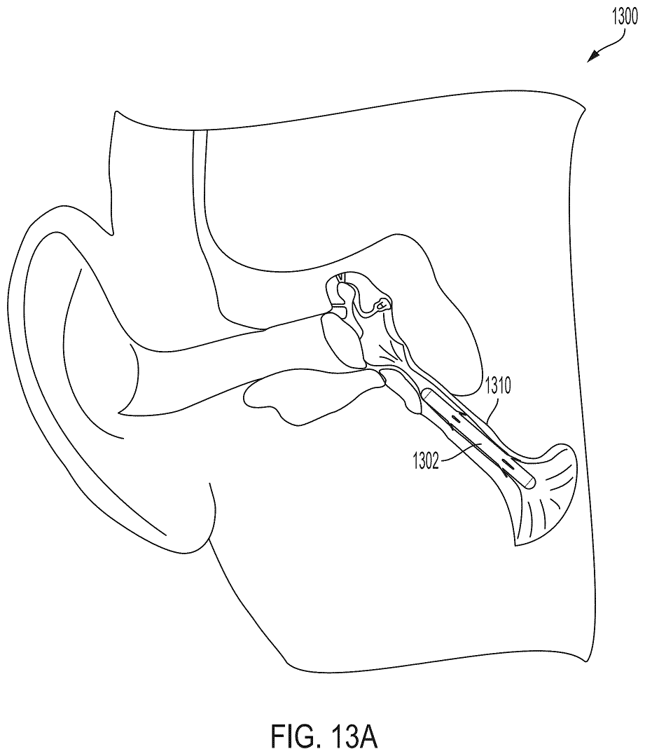

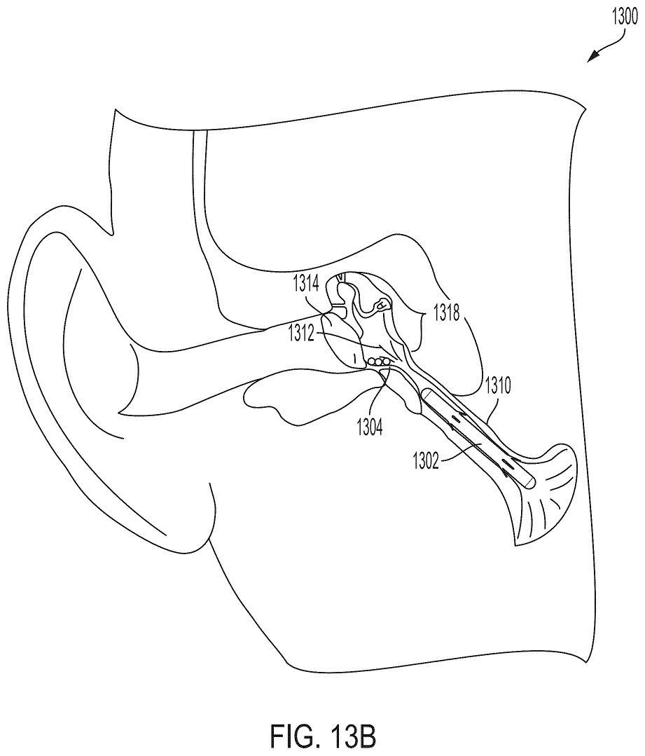

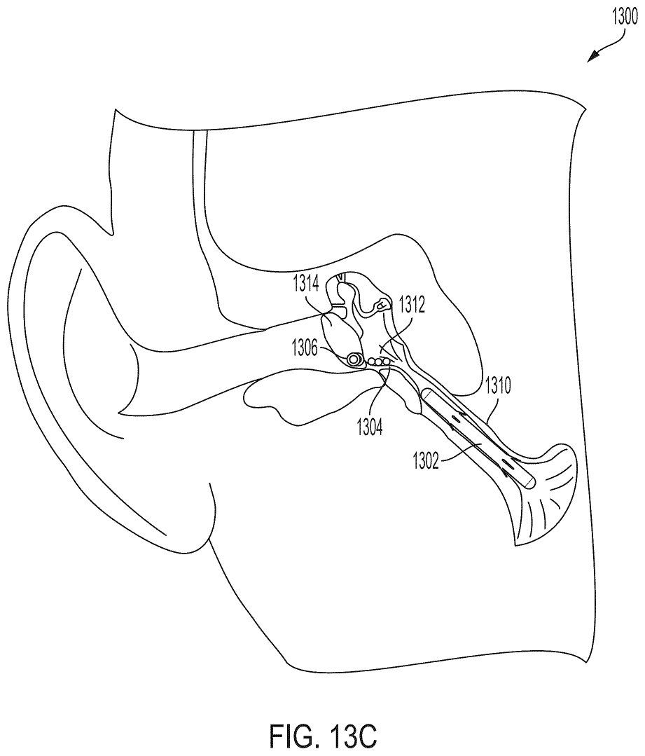

FIG. 13A depicts, in accordance with an embodiment, an implantation 1300 of a eustachian tube therapeutic substance delivery device 1302. The delivery device 1302 is implanted within the eustachian tube 1310.

FIG. 13B depicts, in accordance with an embodiment, an implanted eustachian tube therapeutic substance delivery device 1302 and implanted granules 1304 within the tympanic cavity 1312. The delivery device is implanted within the eustachian tube 1310. The granules 1304 are implanted in the middle ear 1318 via a myringotomy performed in the tympanic membrane 1314.

FIG. 13C depicts, in accordance with an embodiment, an implanted eustachian tube therapeutic substance delivery device 1302, implanted granules 1304 and an implanted ventilation tube 1306. The delivery device stent is implanted within the eustachian tube 1310. The granules 1304 are implanted in the middle ear, within the tympanic cavity 1312. The ventilation tube 1306 is implanted across the tympanic membrane 1314.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

1. A eustachian tube therapeutic substance delivery device comprising:
a body suitable for implantation into a eustachian tube, wherein the body comprises:
a resorbable material, and
a therapeutic substance, wherein the body carries the therapeutic substance so that the therapeutic substance is discharged as the body is resorbed.

2. The eustachian tube therapeutic substance delivery device of claim 1, wherein the body is usable as a stent.

3. The eustachian tube therapeutic substance delivery device of claim 1, wherein the body has a straight cylinder shape.

4. The eustachian tube therapeutic substance delivery device of claim 1, wherein the body has a tapered conical shape.

5. The eustachian tube therapeutic substance delivery device of claim 1, wherein the body has an hourglass shape.

6. The eustachian tube therapeutic substance delivery device of claim 1, wherein the body has a helical shape.

7. The eustachian tube therapeutic substance delivery device of claim 1, wherein the body comprises a mesh.

8. The eustachian tube therapeutic substance delivery device of claim 1, wherein the body includes retention features.

9. The eustachian tube therapeutic substance delivery device of claim 1, wherein the resorbable material is a polymer.

10. The eustachian tube therapeutic substance delivery device of claim 1, wherein the resorbable material is polylactic acid.

11. The eustachian tube therapeutic substance delivery device of claim 1, wherein the resorbable material is polyglycolic acid.

12. The eustachian tube therapeutic substance delivery device of claim 1, wherein the resorbable material is polylactic-co-glycolic acid.

13. The eustachian tube therapeutic substance delivery device of claim 1, wherein the therapeutic substance is impregnated within the body.

14. The eustachian tube therapeutic substance delivery device of claim 1, wherein the therapeutic substance is a coating on the body.

15. The eustachian tube therapeutic substance delivery device of claim 1, wherein the therapeutic substance comprises a steroid.
